(19) United States
(12) Patent Application Publication
(10) Pub. No.: US 2024/0316139 A1
(43) Pub. Date: Sep. 26, 2024

(54) PLANT-BASED ANTIOXIDANT ORAL COMPOSITION

(71) Applicant: NEXIRA, Rouen (FR)

(72) Inventor: Damien GUILLEMET, Rouen (FR)

(73) Assignee: NEXIRA, Rouen (FR)

(21) Appl. No.: 18/694,749

(22) PCT Filed: Sep. 23, 2022
(86) PCT No.: PCT/FR2022/051793

§ 371 Date: Mar. 22, 2024

(30) Foreign Application Priority Data
Mar. 30, 2023 (FR) FR2110157

(51) Int. Cl. A61K 36/87; A61K 9/00; A61K 36/185; A61K 36/28; A61K 36/61; A61P 39/06

(52) U.S. Cl. CPC A61K 36/87; A61K 9/0053; A61K 36/185; A61K 36/28; A61K 36/61; A61P 39/06

(57) ABSTRACT

The invention relates to an antioxidant oral composition comprising a combination of at least two edible plant extracts, said extracts being selected from the group consisting of a grape extract, a witch hazel extract, a clove extract and an immortelle extract.

The invention relates to an antioxidant oral composition comprising a combination of at least two edible plant extracts. The invention further relates to the use of this composition in protecting cells against free radicals and inflammatory processes. In particular, the invention relates to the use of the composition in the treatment and/or prevention of a condition selected from those associated with pro-oxidative and/or inflammatory activities.

It is known that a diet rich in antioxidants can reduce the risk of many diseases, including heart disease certain Antioxidants and cancers. in particular eliminate free radicals from the body's cells and prevent, or at least reduce, damage caused by oxidation. These anti-free radical processes are directly involved in modulating chronic inflammation. To compensate for possible antioxidant deficiencies, the prior art offers dietary supplements comprising antioxidants. These supplements can find applications in sports health (performance and/or recovery), eye and vision health, skin health (sometimes referred to as nutraceuticals), auditory health, and liver health, etc.

At least some of the antioxidants proposed in the prior art are of synthetic origin, that is to say they are produced, for example, by chemical synthesis. The present invention departs from this type of antioxidant and relates to antioxidants derived from plants.

Antioxidants derived from plants are typically identified and/or tested in models using biochemical tests, referred to as in tubo tests, which only involve chemical species, without the presence of live cells, and which do not take into account passage through the intestines or penetration into the body, nor do they take into account the antioxidant's penetration into the cell. In an in vivo approach, in particular in the context of biological applications, biochemical tests are unsatisfactory.

As a result, antioxidants, and in particular their therapeutic, protective and preventive effects, continue to be studied extensively throughout the world.

This invention improves on this situation.

The invention thus relates to an antioxidant oral composition comprising a combination of at least two edible plant extracts, said extracts being selected from the group consisting of grape extract, witch hazel extract, clove tree extract and immortelle extract.

Grape extract is an extract derived from the fruit of the plant, preferably from the seed, and more preferably from VinOseed™. More generally speaking, a purified extract is preferred, i.e. a grape extract (in particular from the seed) with a total polyphenol content of 90% or more. Witch hazel extract is obtained from the aerial part of the plant. Clove tree extract is an extract obtained from the plant's flower bud, preferably clove extract. The immortelle extract is obtained from the aerial part of the plant. The results for these extracts are particularly satisfactory.

In a preferred embodiment, the composition comprises only two edible plant extracts. On the one hand, the synergistic effects observed in this embodiment are highly satisfactory, and on the other hand, it procures raw extract material savings. In other words, in a particular embodiment, the composition of the invention consists of a combination of two edible plant extracts, said extracts being selected from the group consisting of a grape extract, a witch hazel extract, a clove tree extract and an immortelle extract.

In this embodiment, the proportion by weight of each extract is between 20 wt. % and 80 wt. %.

Preferably, the combination of plant extracts is selected from: —a combination of a clove extract and an immortelle extract; —a combination of a clove extract and a grape seed extract, preferably VinOseed™; —a combination of a clove extract and a witch hazel extract; —a combination of a grape seed extract, preferably VinOseed™ and a witch hazel extract. In such a case, the combination of a clove extract and an immortelle extract preferably comprises 50 wt. % clove extract and 50 wt. % immortelle extract; the combination of a clove extract and a grape seed extract, preferably VinOseed™, preferably comprises 50 wt. % clove extract and 50 wt. % grape seed extract; the combination of a clove extract and a witch hazel extract preferably comprises 50 wt. % clove extract and 50 wt. % witch hazel extract; and the combination of a grape seed extract, preferably VinOseed™, and a witch hazel extract comprises 60 wt. % grape seed extract and 40 wt. % witch hazel extract.

The invention further relates to the composition described above, for use in the treatment and/or prevention of a condition in a human or animal, which condition is selected from those associated with pro-oxidative and/or inflammatory activities. Said conditions can in particular include cardiovascular, tissue and joint disorders. Conditions affecting the cardiovascular system include chronic inflammatory diseases such as vascular stiffening, diabetes, etc. Conditions affecting tissues include liver tissue disorders (such as non-bacterial hepatitis, steatosis and inflammatory derivatives, etc.), adipose tissue disorders (hypertrophy and chronic inflammatory status), intestinal tissue disorders (chronic inflammatory conditions such as irritable bowel syndrome, intestinal hyper-permeability, etc.), skin tissue disorders (cell lesions due to exposure to UV rays or pollutants, age- and inflammation-related tissue damage, etc.), hearing tissue disorders (for example tinnitus, age-related hearing loss, etc.), eye tissue disorders (for example age-related macular degeneration), muscle fiber tissue disorders (deleterious effects of free radical generation on muscle fiber, speed of recovery of muscular capacity, etc.). Conditions affecting the joints include in particular osteoarthritis.

The composition may take a form selected from a tablet, cachet, capsule, granule, gelatin capsule, sugar-coated tablet, chewing gum, paste, beverage, syrup and powder. This facilitates oral intake.

The invention further relates to a foodstuff, food supplement, dietary supplement, meal replacement, beverage, beverage supplement, or pharmaceutical product, comprising the composition described above.

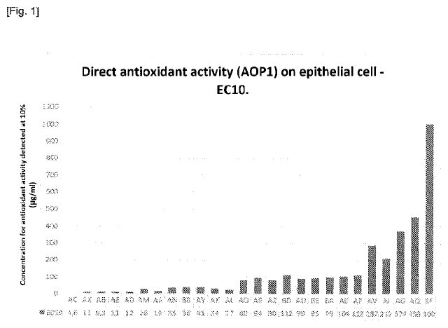

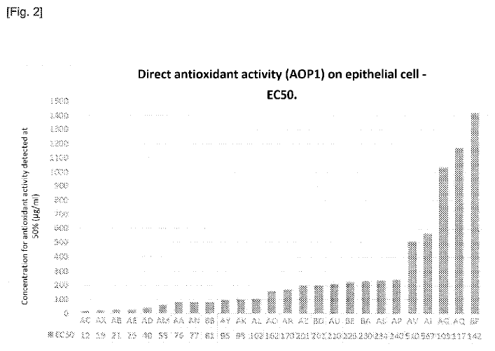

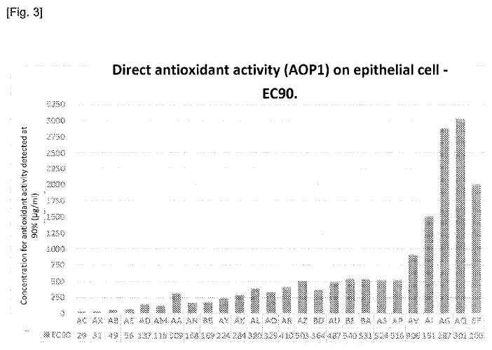

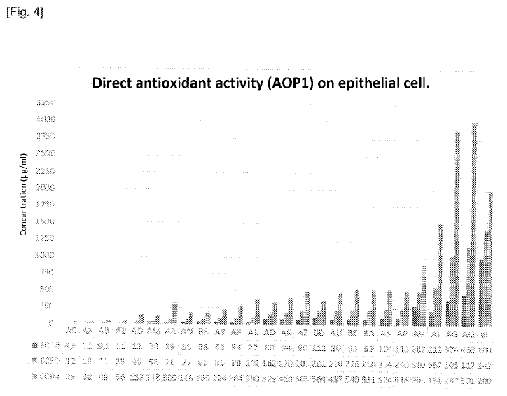

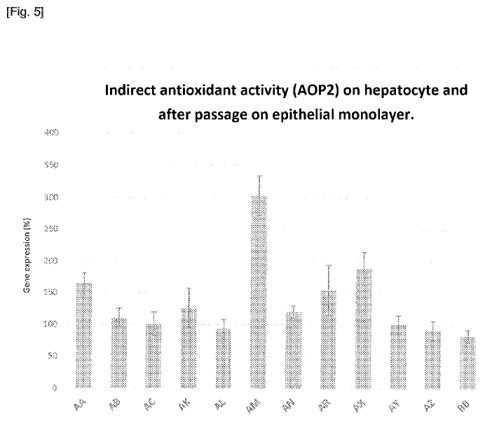

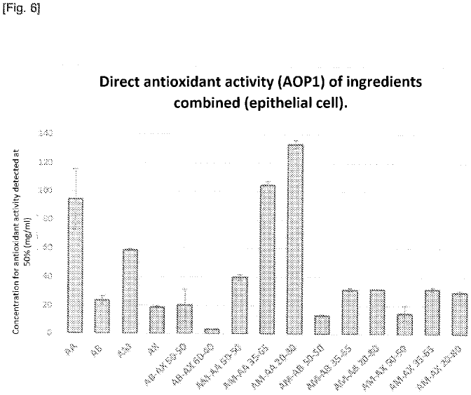

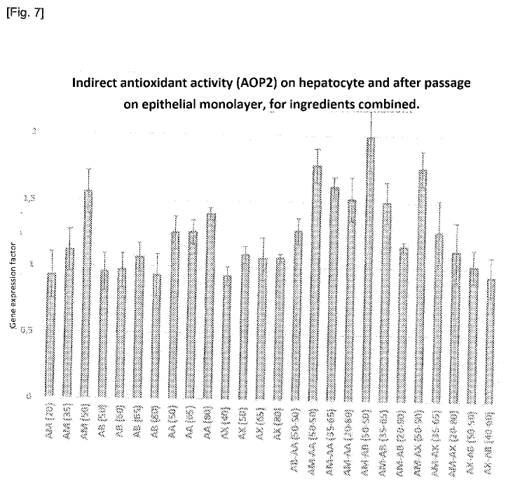

Other features and advantages of the invention will become apparent upon reading the detailed description provided hereinbelow, and from the accompanying drawings in which:

[FIG. 1] shows the results of 10% antioxidant effective concentrations (EC10) of AOP1 from 26 plant extract samples;

[FIG. 2] shows the results of 50% antioxidant effective concentrations (EC50) of AOP1 from 26 plant extract samples;

[FIG. 3] shows the results of 90% antioxidant effective concentrations (EC90) of AOP1 from 26 plant extract samples;

[FIG. 4] shows the results of FIGS. 1 to 3 in a single graph;

[FIG. 5] shows the average relative luminescences of series of Transwell tests representative for AOP2;

[FIG. 6] shows the direct antioxidant effect (AOP1) of sample combinations of the invention on epithelial cells; and

[FIG. 7] shows the indirect antioxidant effect (AOP2) of sample combinations of the invention on epithelial cells.

The figures, tables and the description that follow essentially comprise elements of a definite nature. The 25 figures and tables form an integral part of the description and can thus serve not only to assist with the understanding of the present invention, but also to contribute to the definition thereof, where appropriate.

The term “edible part” of a plant refers to a part of a plant that can be ingested by humans by mouth, i.e. orally or, more generally, via the gastrointestinal tract. Such an “edible part” can also be ingested by an animal. The composition of the invention comprises extracts of edible parts of plants.

Generally speaking, a plant has two main parts: a subterranean part, which in particular includes the root (or tuber or rhizome), and an aerial part, in particular the stem, leaves, buds, fruit and/or flowers.

An edible part of a plant can take a solid or liquid form. It can in particular include leaves, roots, stems, fruit, flowers, and plant exudate or sap, whether or not it has undergone transformation. It can be all or just one or several parts of these elements.

The term “extract” refers to the one or more edible parts of the plant, whether or not they have undergone transformation by an extraction process, present in the composition of the invention. More broadly speaking, the extract can be crude or modified by an extraction process customarily used in the field of food supplements and which retains the desired biological properties.

An extraction process can in particular involve dehydration or cold drying, so that the edible part is transformed into a format suitable for human consumption. Other processes can involve grinding or cutting certain parts of the plants, or an additional solid/liquid extraction using a solvent such as water and/or ethanol for example. Further steps can involve purification by membrane filtration or resin absorption or by ion exchange. Plant extracts can thus take in particular a powder or granule form. Processes can also include washing, disinfection, bleaching, drying and/or cooking plant parts. Extracts can therefore also take the form of a suspension, an aqueous solution or a dispersion for example. Moreover, extracts can take the form of “plant totum”, i.e. a whole plant or part thereof, that has been dehydrated by drying and then ground.

The composition of the invention can take various forms suitable for oral ingestion. In particular, the composition can take the form of tablets, gelatin capsules, a powder, a liquid or a syrup. The composition of the invention can thus comprise pharmaceutically acceptable and/or agri-food-compatible carrier substances. The composition can further comprise ingredients such as diluents, adjuvants, excipients, preservatives, fillers, disintegrants, wetting agents, emulsifiers, anti-caking agents, suspending agents, flavor intensifiers, aromatic agents, fragrances, antibacterial agents, yeast inhibitors, lubricants and dispersants. Formulation techniques are in particular described in Remington: The Science and Practice of Pharmacy, 19th Edition, ISBN-13: 978-0912734040 or in Conception des compléments alimentaires: Marché, développement, réglementation et efficacité, ISBN-13: 978-2743022211.

The antioxidant composition of the invention is primarily intended for the dietary supplement market. The invention in particular targets foodstuffs, food supplements, dietary supplements, beverages and beverage supplements. However, the invention also targets the pharmaceutical market. It thus targets a pharmaceutical product in its usual form.

In the context of biological applications, in tubo/biochemical tests are not the most representative, and cellular models should be used to best anticipate the potential results in humans. To this end, the Applicant has used a system that measures antioxidant activity inside the cell, in order to take into account the ingredient's ability to penetrate the cell and protect it from deleterious pro-oxidant processes and the inflammatory consequences thereof.

Therefore, in order to study the antioxidant effect of an extract and/or a combination of extracts, the Applicant has used a technology known as LUCS (Light-Up Cell System). This technology is described in particular in the European patent document EP2235505 or in the articles by Gironde, C., Rigal, M., Dufour, C., & Furger, C. (2020). AOP1, a new live cell assay for the direct and quantitative measure of intracellular antioxidant effects. Antioxidants, 9(6), 471 .; and Derick S. et al. (2017), LUCS (Light-Up Cell System), a universal high throughput assay for homeostasis evaluation in live cells, Scientific Reports—Nature, 7, p. art. 18069 [11 p.]. ISSN 2045-2322.

LUCS technology is based on a fluorescence assay and measures the effect of antioxidants on live cell models. More specifically, the technology uses variations in the fluorescence intensity of thiazole orange (TO), a nuclear biosensor belonging to the asymmetric cyanine family, to establish the cell's homeostasis status. This allows cellular antioxidant effects to be qualified.

Generally speaking, the biosensor (TO) is added to the cell culture medium, and becomes fluorescent after association with the cell nucleic acids. The TO is activated by incident light at a wavelength of 480 nm, then relaxes while transferring part of its energy to the intracellular oxygen. This generates intracellular singlet oxygen, which leads to the production of reactive oxygen species (ROS), in particular including superoxide anion (O2·−) and hydroxyl radical (OH·). The antioxidant effect is measured by the ability of the sample tested to neutralize this intracellular ROS production. More specifically, the intracellular antioxidant effect is revealed by a time delay in the fluorescence increased triggered by the photo-activation of the biosensor under successive light flashes.

The Applicant has followed a more complex protocol to identify the antioxidant activity of the extracts or combinations of extracts of the invention. This protocol involves studying the bioavailability of plant extracts, i.e. before and after passing through the intestines. Moreover, this protocol also distinguishes between so-called direct antioxidant activity (AOP1, Anti-Oxidant-Power 1) and so-called natural antioxidant activity (AOP2, Anti-Oxidant-Power 2). AOP1 corresponds to the extracts' ability to directly scavenge intracellular ROS, whereas AOP2 corresponds to the extracts' ability to stimulate the cell's natural antioxidant defense pathway by activating the ARE/Nrf2 gene.

AOP2 technology is described in particular in the article by Furger, C. (2021). Live Cell Assays for the Assessment of Antioxidant Activities of Plant Extracts. Antioxidants, 10(6), 944.

More particularly, the first activity tested was carried out in a model using cell lines representative of the intestinal barrier, which is the first to be crossed by an orally-ingested ingredient (AOP1). In order to further improve biological representativeness in the context of an orally-ingested ingredient, the Applicant has used an innovative model to simulate the passage of the ingredient through an intestinal barrier, and then to test the passing products on liver cells, representative of the first organ through which the ingredient passes once it has reached the bloodstream (this model is used in the case of AOP2 results).

The Applicant therefore firstly studied the AOP1 of a large number of plant extracts, before and after passing through the intestines, to identify candidate extracts with enhanced antioxidant activity. The

Applicant then studied the AOP2 of the candidates retained. The Applicant subsequently discovered, not without surprise, that compositions comprising a combination of extracts selected from among those retained, resulted in antioxidant properties far superior to compositions of the prior art. It would appear that a synergistic effect is behind the drastically increased antioxidant activity. The compositions according to the invention thus offer unprecedented properties.

### EXPERIMENTAL PART 1

The AOP1 of 28 plant extracts was studied on epithelial cells.

The extracts are given in Table 1 below:

### [Table 1]

TABLE 1
Extracts

| Code | Latin name | Common name | Part of plant |
|---|---|---|---|
| AA | Helichrysum italicum | Immortelle | aerial part |
| AB | Vitis vinifera | VinOseed 40% | seed |
| AC | Vitis vinifera | VinOseed So free | seed |
| AD | Vitis vinifera | VinOgrape Plus | various fruit |
| | | | parts |
| AE | Vitis vinifera | VinOgrape Prestige | various fruit |
| | | | parts |
| AG | Olea europaea | Oli Ola 3% clear Bio | fruit |
| AI | Vaccinium macrocarpon | Cranberry - Juice | fruit |
| AJ | Malpighia emarginata | Organic green acerola | fruit |
| AK | Ribes nigrum | Blackcurrant | leaf |
| AL | Cinnamomum verum | Cinnamon | bark |
| AM | Syzygium aromaticum | Clove | flower |
| AN | Vitis vinifera | Grape | leaf |
| AO | Vitis vinifera | Grape | seed |
| AP | Sambucus nigra | Elder - Juice | fruit |
| AQ | Glycyrrhiza glabra | Liquorice | Root |
| AR | Rosmarinus officinale | Rosemary | Leaves |
| AS | Thymus vulgaris | Thyme | Leaves |
| AU | Ocimum tenuiflorum | Holy basil | leaf |
| AV | Andrographis paniculata | Green chiretta | aerial parts |
| AW | Undaria pinnatifida | Wakame | kelp |
| AX | Hamamelis virginiana | Witch hazel | aerial parts |
| AY | Ilex paraguariensis | Yerba mate | Worksheet |
| AZ | Melissa officinalis | Lemon balm | Leaf |
| BA | Salvia officinalis | Sage | Leaf |
| BB | Vaccinium myrtillus | Blueberry - Juice | Fruit |
| BD | Ficus Carica | Fig tree | Leaf |
| BE | Morus Nigra | Mulberry tree | Leaf |
| BF | Lonicera Caerulea | Fly honeysuckle - | Fruit |
| | | Juice | |

The antioxidant effect AOP1 (direct effect) prior to passage through the intestines was achieved using a human intestinal epithelial cell model. The cells used in this case were Caco-2 cells, which form a human tumor cell line (immortalized cells) of intestinal origin isolated from a colon adenocarcinoma.

Extracts took powder form. They were weighed (250 mg), then dissolved in 5 ml of fetal calf serum-free (FCS-free) DMEM cell culture medium. Samples (50 mg/ml) of the solution were vortexed, then centrifuged at 8,700 rpm for 10 minutes. Supernatants were aliquoted and kept at −20° C. All samples were kept at 4° C. until the day of the AOP1 assay and diluted in an FCS-free DMEM cell culture medium.

The sample BF was prepared from a crude sample using the following protocol: grinding in a blender for 5 for minutes, magnetic stirring 30 minutes, centrifugation at 5,000 G for 10 minutes, then collection of the supernatant. The dry matter of AI and BF was weighed: AI=463 mg/ml; BF=145 mg/ml.

For the study using human CaCo2 cells, these cells were seeded into 96-well plates at a density of 30,000 cells/well in a DMEM medium with fetal calf serum (FCS) as a medium supplement. The plates were stored in an incubator for 24 hours at 37° C./5% CO₂.

The cells were then incubated in the presence of samples (6 concentrations obtained by a series of 16 log(3) dilutions) for 4 hours at 37° C./5% CO₂. Experiments were carried out in an FCS-free DMEM medium. At least two independent experiments were carried out, each in triplicate wells.

After incubation for 4 hours, the cells were treated with the fluorescent biosensor for 1 hour and fluorescence was measured (RFU at 535 nm) using a procedure involving repetitive application of 480 nm LED light (20 iterations) to the entire 96-well plate. The kinetic profiles were recorded. Each monograph sample showed the crude RFU values recorded during the kinetics for each concentration tested, before and after subtraction of blank values (non-specific fluorescence). Normalized data (°/0 age of control values) and dose-response curves were also studied. The cellular antioxidant index (AOP index) was calculated from normalized kinetic profiles according to the formula:

$$AOP index ( \% ) = 100 - 100 ( {}_{0} {\int }^{20} {RFU}_{sample} / {}_{0} {\int }^{20} {RFU}_{control} ) \qquad [ Math . 1 ]$$

The dose-response curves, obtained by compiling AOP indices according to the log(10) of the sample concentration, were submitted to a sigmoid fit according to the formula:

$$AOP index ( \% ) AOP {index}_{min} + ( AOP {index}_{max} - AOP {index}_{min} ) / ( 1 + {10}^{( Log ( EC 50 ‐ SC ) * HS )} ) \qquad [ Math . 2 ]$$

where SC is equal to the sample concentration and HS is equal to the Hill slope. The EC50 (50% effective concentration), EC10 (10% effective concentration) and EC90 (90% effective concentration) were then evaluated according to the fit.

The results were studied in monographs. Each data set corresponds to an experiment representative. In all cases, the two independent experiments gave similar results.

All but two samples (AJ and AW) showed EC values.

FIG. 1 shows the results of the 26 samples, classed according to the calculated EC10 values.

EC10 quartile analysis:
Q1 (lowest EC10 value)=(AC; AB; AX; AE; AD; AA; AL)
Q2 (lowest EC10 value)=(AM; AK; AN; BB; AY)

FIG. 2 shows the results of the 26 samples, classed according to the calculated EC50 values.

EC50 quartile analysis:
Q1 (lowest EC50 value)=(AC; AX; AB; AE; AD; AM; AA)
Q2 (lowest EC50 value)=(AN; BB; AY; AK; AL)

FIG. 3 shows the results of the 26 samples, classed according to the calculated EC90 values.

EC90 quartile analysis:
Q1 (lowest EC90 value)=(AC; AX; AB; AE; AM; AD; AN)
Q2 (lowest EC90 value)=(BB; AY; AK; AA; AO; BD)

FIG. 4 shows the EC10, EC50 and EC90 results for the 26 samples in a single graph.

### Experimental Part 2

Based on the results obtained in the first experimental part, 12 samples were selected: AA, AB, AC, AK, AL, AM, AN, AR, AX, AY, AZ, and BB.

Table 2 shows the 12 samples selected.

TABLE 2
12 samples selected

| Sample code | Common name | Part of plant |
|---|---|---|
| AC | VinOseed So free | seed |
| AX | Witch hazel | aerial part |
| AB | VinOseed 40% | seed |
| AM | Clove | flower |
| AA | Immortelle | aerial part |
| AN | Grape F | leaf |
| BB | Blueberry | Fruit |
| AY | Yerba mate | Leaf |
| AK | Blackcurrant | leaf |
| AL | Cinnamon | bark |
| AR | Rosemary | Leaves |
| AZ | Lemon balm | Leaf |

The aim of the second experimental part was to subject all selected samples to an intestinal barrier test in order to collect the basolateral compartments and assess the residual antioxidant activities of the AOP1 and AOP2 technologies. Liver cells (human HepG2 model) were chosen to assess these activities, since the liver represents the first organ targeted after the ingredients or metabolites are transferred into the blood (portal vein).

For comparison with experimental part 1, AOP1 activity was also measured for each sample in direct contact with the same liver cells (HepG2).

The second experimental part was divided into two main tasks:

Task 1: conditioning the 12 samples using an intestinal transport test performed on the same human epithelial cell model already used for the first part (but in a differentiated confluent monolayer version so as to rebuild the physiological barrier as present in the intestine and which enables nutrients to be filtered/absorbed into the body).

Task 2: testing the 12 basolateral compartments collected by the AOP1 and AOP2 technologies on human HepG2 liver cells determine their complete to antioxidant profiles.

To study the antioxidant effect AOP1 after passage through the intestines, a bioavailability study of the extracts on Caco-2 cells can be carried out. This study allows the fraction of the product (or the molecular part) that is assimilated by the human body after administration by mouth to be isolated. For example, when a product comprises an active ingredient, it may be the intact fraction of this active ingredient that reaches the bloodstream. In other cases, this fraction may consist of a combination of molecules from the product, or may be made up of molecules that have undergone biochemical transformation or cellular metabolization.

The Caco-2 cells form a human tumor cell line (immortalized cells) of intestinal origin isolated from a colon adenocarcinoma.

Under certain culture conditions, these cells have the capacity to spontaneously differentiate into intestinal cells organized in a contiguous, polarized monolayer to form an epithelium mimicking a functional intestinal barrier. The epithelium can in particular have microvilli, tight junctions, intestinal-specific transporters, and enzymes of the metabolization process, cf. Pinto M. et al, Enterocyte-like differentiation and polarization of the human colon carcinoma cell line CACO-2 in culture, Biol Cell, 1983, 47:323-30, and Hidalgo I J. et al., Characterization of the human colon carcinoma cell line (Caco-2) as a model system for intestinal epithelial permeability, Gastroenterology, 1989, 96 (3):736-49.

Tests on Caco-2 cells allow intestinal permeability to be applied to a product. In other words, tests on Caco-2 provide information on a compound's ability to cross the intestinal wall and, where appropriate, its biochemical transformation, before entering the bloodstream and lymph circulation and being distributed throughout the body.

Here, a Caco-2 permeability test was used to obtain the bioavailable fractions of the extracts of the invention. This was a monolayer isolated compartment insert.

Caco-2 cells were grown for this purpose on a microporous membrane placed in individual culture chambers. After a culture period of around 20 to 25 days, the cells formed a differentiated monolayer isolating the upper and lower compartments of the culture chamber.

Once the bioavailable fraction was isolated, the AOP1 antioxidant effect of this fraction could be studied. More specifically, use of the bioavailable fraction makes the assessment biologically significant. In particular, it drastically increases in vivo representativeness.

The AOP2 antioxidant effect of the extracts and their combinations could then be studied on the human HepG2 liver cells to determine the complete antioxidant profile (see below after the AOP1 study).

Frozen fractions of the samples used for the first phase were thawed and used for Task 1.

As mentioned above, the LUCS approach is based on the production of cellular radical species following the addition of a photo-inducible fluorescent nucleic acid biosensor to the culture medium. Under the effect of light, the cellular biosensor triggers the production of singlet oxygen, which in turn triggers the production of ROS (Reactive Oxygen Species) in a biochemical cascade linked to an increase in emitted fluorescence.

The ARE/Nrf2 live cell assay was used to determine the AOP2. This is a gene-based approach that measures the sample's ability to activate the ARE (Antioxidant Response Element) DNA promoter sequence following secretion, from the nucleus, of the Nrf2 transcription factor from the cytosolic Keapl/Nrf2 complex. This genome pathway (often referred to as the “Natural Cellular Antioxidant Defense”) is known to enhance cells' ability to adapt to oxidative and other stresses. Further details are described in the publication Ma (2013), Role of Nfr2 in oxidative stress and toxicity, Annual Review of Pharmacology & Toxicology, 53: 401. In HepG2-Nrf2 cells, the ARE promoter sequence is coupled to the expression of the luciferase enzyme which, in the presence of luciferin, catalyzes the production of the luminescent oxyluciferin compound.

The intestinal barrier assay is a permeability comprises a testing system. The system typically Transwell® insert marketed by Merck. The insert comprises a microporous membrane on which Caco-2 cells are grown as a monolayer. The microporous membrane/Caco-2 cell assembly separates an apical compartment from a basolateral compartment. The microporous membrane is typically arranged in a plurality of wells. Each well has a certain diameter (for example 24 mm) and a microporous membrane bottom with pores (for example 0.4 pm). Each well is seeded with a defined cell density (for example 100,000 cells/well). The culture medium is changed regularly to obtain a cell monolayer, cf. Reboul et al., Lutein transport by Caco-2 TC-7 cells occurs partly by a facilitated process involving the scavenger receptor class B type I (SR-BI), Biochem J, 2005, 387 (Pt 2):455-61.

The apical and basolateral compartments were respectively filled with culture medium. The cells can then be exposed to the plant extracts for a set period of time. The respective cells and culture media from the apical and basolateral compartments are harvested and analyzed. Optionally, the cells and respective culture media from the apical (apical medium) and basolateral (basolateral medium) compartments are frozen at −80° C. for subsequent experiments. The use of such a system provides access to both the apical side (the side above the cells, mimicking the intestinal lumen) and the basal side (the side below the cells, mimicking the internal environment) of the intestinal epithelium. This makes it possible to evaluate the product exchanges between the two sides (apical/basal). The Caco-2 cell permeability assay can in particular be used to investigate the permeability of the intestinal epithelium to a product of interest, the expected bioavailability in humans, the screening and selection of products of interest, the absorption kinetics of the intestinal system, or the biological study of absorption mechanisms.

After exposing Caco-2 cells to plant extracts or to combinations of plant extracts added to the apical medium, the basolateral medium comprising the bioavailable fraction of the extracts or the combinations thereof can then be harvested. It should be noted that the basolateral medium can also comprise metabolites resulting from the specific exposure of Caco-2 cells to the plant extracts. The basolateral medium is typically harvested and stabilized by freezing at −80° C.

The Applicant has thus assessed the bioavailability of the selected plant extracts in order to subsequently evaluate the antioxidant effect of the bioavailable fractions.

This case involved the use of human CaCo2 cells assayed in Transwell-type 12-cluster Corning polycarbonate culture plates (pore=3 μm) in a DMEM culture medium supplemented with 20% FCS (Fetal Calf Serum) at a density of 760,000 cells per well (500 μI). Cells were grown for 21 days at 37° C./5% CO2. The culture medium was replaced every day (except on weekends). The TEER (Trans Epithelial Electrical Resistance) value was measured regularly with a dedicated ohmmeter to assess the integrity and stability of the cell barrier. On day 21, the TEER value was measured and the Transwell plates were treated with the samples at 10 mg/ml (with the exception of sample AA, which was prepared at 1 mg/ml) for 4 hours. The apical compartment (500 μI) and basolateral compartment (1,500 μI) were collected at the end of the incubation time, then aliquoted and kept at −20° C. for further experiments. Two independent experiments were carried out for each sample.

The AOP1 and AOP2 studies were performed on human HepG2 cells. The cells were seeded into 96-well plates at a density of 75,000 cells/well in a DMEM medium supplemented with fetal calf serum (FCS). The plates were incubated for 24 hours at 37° C./5% CO2.

The cells were then incubated in the presence of the samples (6 concentrations obtained per log2 dilution series) for 4 hours (AOP 1) or 17 hours (AOP 2) at 37° C./5% CO2. Experiments were carried out in an FCS-free DMEM medium. One experiment in triplicate wells (AOP 1) and two independent experiments in duplicate wells (AOP 2) were carried out.

For the AOP1 assay, the cells were treated after 4 hours of incubation with the fluorescent biosensor for 1 hour and fluorescence was measured (RFU at 535 nm) after repetitive LED exposure at 480 nm (20 iterations) of the entire 96-well plate. The kinetic profiles were recorded. Monographs presenting the crude RFU values recorded during the kinetics for each concentration tested were studied. Dose-response curves were also studied. The cellular antioxidant index (AOP index) was calculated from normalized kinetic profiles according to the formula:

$$AOP index ( \% ) = 100 - 100 ( {}_{0} {\int }^{15} {RFU}_{sample} / {}_{0} {\int }^{15} {RFU}_{control} ) \qquad [ Math . 3 ]$$

The dose-response curves, obtained by compiling AOP indices according to the logarithm(10) of the sample concentration, were submitted to a sigmoid fit according to the formula:

$$AOP index ( \% ) AOP {index}_{min} + ( AOP {index}_{max} - AOP {index}_{min} ) / ( 1 + {10}^{( Log ( EC 50 ‐ SC ) * HS )} ) \qquad [ Math . 4 ]$$

where SC is equal to the sample concentration and HS is equal to the Hill slope. The EC50, EC10 and EC90 values were then calculated.

Due to low sample availability for the basolateral compartment, a single AOP1 experiment was performed in triplicate.

For the AOP2 assay, the cells were treated after 17 hours of incubation with a mix (BPS Bioscience, USA) comprising lysis a cell solution and luciferin (luciferase substrate) for 40 minutes. Luminescence was read on a Varioskan reader (marketed by Fischer). The luminescence values (RLU) revealed luciferase gene expression following ARE gene expression. The results are presented as Fold Increase (FI) in the control values at t=30 minutes, according to the following formula:

$$Fold Increase ( FI ) = {RLU}_{sample} / {RLU}_{control} \qquad [ Math . 5 ]$$

Monographs presenting the FI and, assuming that the minimum and maximum asymptotes have been considered by sigmoid fit, the dose-response curve is studied. The EC10, EC50, EC90 values and the Hill slopes are calculated from the sigmoid fit according to the following formula:

$$FI = {FI}_{min} + ( {FI}_{max} - {FI}_{min} ) / ( 1 + {10}^{( Log ( EC 50 ‐ SC ) * HS )} )$$

where SC is equal to the sample concentration and HS is equal to the Hill slope. If the sigmoid fit fails, extrapolated EC10 and EC50 values are evaluated.

The TEER (Trans Epithelial Electrical Resistance) is then analyzed. Table 3 shows the TEER measured between the apical compartment and basolateral compartment before and after incubation of each selected sample.

TABLE 3
TEER epithelial resistance test

| | TEER before adding | TEER after adding |
|---|---|---|
| Sample | the sample | the sample |
| AA | 669 | 520 |
| AB | 931 | 628 |
| AC | 839 | 570 |
| AK | 720 | 500 |
| AL | 1021 | 870 |
| AM | 942 | 592 |
| AN | 1040 | 925 |
| AR | 921 | 884 |
| AX | 981 | 940 |
| AY | 931 | 845 |
| AZ | 1033 | 800 |
| BB | 937 | 780 |
| Negative control | 24 | |

The AOP1 and AOP2 on the pre-apical and basolateral compartments of the HepG2 cells are then measured.

With the exception of sample AA, each of the 12 samples gave a result for EC50. Table 4 shows a comparison between the results obtained on CaCo2 cells (cf. first experimental phase) and the results obtained on HepG2 cells (second experimental phase):

TABLE 4
Comparison of EC50 CaCo vs. HEPG2

| | EC50 AOP1 | EC50 AOP1 |
|---|---|---|
| Sample | CACO2 (μg/ml) | HEPG2 (μg/ml) |
| AA | 76.25 | NA |
| AB | 20.96 | 16.76 |
| AC | 11.62 | 15.74 |
| AK | 97.77 | 143.30 |
| AL | 102.10 | 52.09 |
| AM | 57.78 | 46.73 |
| AN | 76.75 | 59.78 |
| AR | 169.90 | 174.70 |
| AX | 18.58 | 23.00 |
| AY | 95.43 | 131.40 |
| AZ | 200.50 | 248.20 |
| BB | 80.70 | 308.20 |

AOP1: None of the basolateral samples showed conventional antioxidant effects

AOP1 (direct effect through neutralization of free radicals). It is conceivable that the 4-hour incubation period was insufficient. However, an increase in the incubation time to 17 hours did not change the outcome. It can be seen that the kinetic profiles of the basolateral samples showed a slight shift to the left compared with the control curve. This phenomenon is particularly noticeable for samples AC, AK, AL, AR and AZ.

AOP2: FIG. 5 shows the data grouped from 2 independent Transwell experiments (series 1 and 2). Error bars are indicated and come from quadruplicate measurements). The basolateral compartments were derived from 4 hours of incubation with 10 mg/mL samples, with the exception of AA (1 mg/mL).

Table 5 summarizes the data available for the 12 ingredients after experimental part 2.

TABLE 5
Available data

| | Bioavailable | Cellular |
|---|---|---|
| Antioxidant activities | fraction | origin |
| AOP 1 (direct, phase I) | No | Intestinal |
| AOP 1 (direct) | No | Liver |
| AOP 2 (defense activation) | Yes | Liver |

### Experimental Part 3

One of the Applicant's approaches was to consider that the plant extracts can have very different activities, even within the same model, and that they should be selected for their potential. Moreover, the opportunity to couple them can lead to significant optimizations of biological antioxidant potential. The applicant has identified specific combinations of plant extracts assembled in such a way as to provide synergistic biological antioxidant protection (i.e. superior to the ingredients tested separately). These combinations can be used as an orally-administered food supplement and for cellular protection applications (protecting against free radicals and inflammatory processes) for benefits such as sports health (performance and recovery), eye health and vision, skin health also known as nutraceuticals, auditory health, liver health, etc. Moreover, the selected combinations can be used alone or in association with other functional or technical ingredients. The target population can be both human and animal.

Firstly, some of the samples were sorted by activity. Table 6 shows the sorting carried out:

TABLE 6
Classification by activity

| | AOP2 | Stand | AOP2 | AOP 1 | AOP 1 |
|---|---|---|---|---|---|
| Name | Bioavail | Dev | Direct | Epith. | Hepat. |
| Clove | 292.4 | 30.3 | ** | 57.8 | 46.7 |
| Witch hazel | 182.4 | 25 | | 18.6 | 23 |
| Immortelle | 157.4 (×5) | 16.9 | | 76.3 | NA |
| Rosemary | 149.3 | 38.3 | ** | 169.9 | 174.7 |
| Blackcurrant | 121.9 | 29.8 | ** | 97.8 | 143.3 |
| Grape F | 116 | 9.5 | * | 76.8 | 59.8 |
| VinOseed 40% | 106.3 | 14.2 | | 21 | 16.8 |
| Cinnamon | 90.3 | 14.5 | * | 102.1 | 52.1 |

Combinations of samples were then studied.

FIG. 6 shows the direct antioxidant effect (AOP1) of sample combinations on epithelial cells.

Considerable synergy was observed for the Clove/Immortelle 50/50, Clove/Vinoseed 50/50, Clove/Witch Hazel 50/50 and Vinoseed/Witch Hazel 60/40 combinations.

FIG. 7 shows the indirect antioxidant effect (AOP2) of sample combinations on epithelial cells.

Considerable synergy was observed for the Clove/Vinoseed 50/50 combination. The Clove/Immortelle 50/50 combination showed partial synergy. A rather linear effect was observed for the Clove/Witch Hazel 50/50 combination.

It appears that Clove, Witch Hazel, Immortelle and Grape Seed extract (VinOseed) combinations were the most effective, with significant synergies.

In order to further investigate the synergistic effect between the extracts, the Applicant has calculated the synergies of the combinations. The results are shown in Tables 7 and 8 below.

TABLE 7
Synergistic effect of extract combinations on AOP1 activity

| | | Theoretical | EC₅₀ | |
|---|---|---|---|---|
| | | EC₅₀ | Observed | Var. |
| Extract combination | %/% | (μg/ml) | (μg/ml) | (%) |
| S. aromaticum/H. italicum | 50/50 | 76.20 | 39.64 | −47.98 |
| | 35/65 | 81.55 | 104.20 | 27.78 |
| | 20/80 | 86.90 | 133.10 | 53.17 |
| S. aromaticum/H. virginiana | 50/50 | 38.47 | 14.62 | −61.99 |
| | 35/65 | 32.49 | 31.60 | −2.75 |
| | 20/80 | 26.52 | 29.30 | 10.47 |
| S. aromaticum/V. vitifera 5 | 50/50 | 40.76 | 12.74 | −68.74 |
| | 35/65 | 35.48 | 31.30 | −11.77 |
| | 20/80 | 30.19 | 31.70 | 4.99 |
| H. virginiana/V. vitifera 5 | 50/50 | 20.86 | 20.07 | −3.76 |
| | 40/60 | 21.31 | 3.30 | −84.52 |

Table 7 shows a comparison of AOP1 EC₅₀ calculated from dose-response obtained with different combinations of extracts on CaCo2 cells. The theoretical EC₅₀ has been calculated taking into account the proportional contribution of the individual activities of each extract. Var. (%) is the percentage variation of the two Theoretical EC₅₀ and Observed EC₅₀ values. Variations with negative values show a marked synergistic effect.

TABLE 8
Synergistic effect of extract combinations on AOP2 activity

| | | FI | Theoretical | Var. |
|---|---|---|---|---|
| Extract combination | %/% | observed | FI | (%) |
| S. aromaticum/H. italicum | 50/50 | 1.77 | 1.82 | −2.43 |
| | 35/65 | 1.61 | 1.38 | 16.75 |
| | 20/80 | 1.52 | 1.33 | 14.32 |
| S. aromaticum/H. virginiana | 50/50 | 1.76 | 1.66 | 6.32 |
| | 35/65 | 1.28 | 1.18 | 8.31 |
| | 20/80 | 1.13 | 1.00 | 12.93 |
| S. aromaticum/V. vitifera 5 | 50/50 | 2.00 | 1.52 | 31.41 |
| | 35/65 | 1.50 | 1.18 | 27.13 |
| | 20/80 | 1.17 | 0.86 | 35.31 |
| H. virginiana/V. vitifera 5 | 50/50 | 1.02 | 1.05 | −2.63 |
| | 40/60 | 0.94 | 0.90 | 4.37 |

Table 8 shows in particular the synergistic effects on ARE/Nrf2 gene expression in hepatocytes by combinations at post-intestinal barrier compartments. The FI Observed corresponds to the measurement of the increase in gene expression factor (FI) for each combination; the theoretical FI corresponds to the sum of the measurements of the increase in gene expression factor (FI) for each of the two extracts taken independently; Var. (%) is the percentage variation between the observed and theoretical FI values. The variations with a positive value show a marked synergistic effect.

The particularly noteworthy combinations are:
Clove/Grape Seed—50/50;
Clove/Witch Hazel—50/50;
Clove/Immortelle—50/50; and
Grape Seed/Witch Hazel—60/40.

It goes without saying that slightly different ratios can be used for the invention.

The above preferred combinations can also be conceivably combined with other samples, particularly those listed in Table 7, such as sage or blackcurrant.

1. Antioxidant oral composition comprising a combination of at least two edible plant extracts, said extracts being selected from the group consisting of grape extract, witch hazel extract, clove tree extract and immortelle extract.

2. Composition according to claim 1, wherein the grape extract is an extract derived from the fruit of the plant, preferably from the seed, and more preferably from VinOseedTM.

3. Composition according to claim 1, wherein the witch hazel extract is obtained from the aerial part of the plant.

4. Composition according to claim 1, wherein the clove tree extract is an extract obtained from the plant's flower bud, preferably clove extract.

5. Composition according to claim 1, wherein the immortelle extract is obtained from the aerial part of the plant.

6. Composition according to claim 1, comprising only two edible plant extracts.

7. Composition according to claim 6, wherein the proportion by weight of each extract is between 20 wt. % and 80 wt. %.

8. Composition according to claim 7, wherein the combination of plant extracts is selected from: —a combination of a clove extract and an immortelle extract; —a combination of a clove extract and a grape seed extract, preferably VinOseed™; —a combination of a clove extract and a witch hazel extract; —a combination of a grape seed extract, preferably VinOseed™ and a witch hazel extract.

9. Composition according to claim 8, wherein the combination of a clove extract and an immortelle extract comprises 50 wt. % clove extract and 50 wt. % immortelle extract; the combination of a clove extract and a grape seed extract, preferably VinOseed™, comprises 50 wt. % clove extract and 50 wt. % grape seed extract; the combination of a clove extract and a witch hazel extract comprises 50 wt. % clove extract and 50 wt. % witch hazel extract; and the combination of a grape seed extract, preferably VinOseed™, and a witch hazel extract comprises 60 wt. % grape seed extract and 40 wt. % witch hazel extract.

10. Composition according to claim 7, for use in the treatment and/or prevention of a condition selected from a condition related to pro-oxidative and/or inflammatory activities in a human or animal.

11. Composition according to claim 7, taking a form selected from a tablet, cachet, capsule, granule, gelatin capsule, sugar-coated tablet, chewing gum, paste, beverage, syrup and powder.

12. Foodstuff, food supplement, dietary supplement, meal replacement, beverage, beverage supplement, or pharmaceutical product, comprising the composition according to claim 7.

13. Composition according to claim 1, wherein the combination of plant extracts is selected from: —a combination of a clove extract and an immortelle extract; —a combination of a clove extract and a grape seed extract, preferably VinOseed™; —a combination of a clove extract and a witch hazel extract; —a combination of a grape seed extract, preferably VinOseedTM and a witch hazel extract.

14. Composition according to claim 13, wherein the combination of a clove extract and an immortelle extract comprises 50 wt. % clove extract and 50 wt. % immortelle extract; the combination of a clove extract and a grape seed extract, preferably VinOseed™, comprises 50 wt. % clove extract and 50 wt. % grape seed extract; the combination of a clove extract and a witch hazel extract comprises 50 wt. % clove extract and 50 wt. % witch hazel extract; and the combination of a grape seed extract, preferably VinOseed™, and a witch hazel extract comprises 60 wt. % grape seed extract and 40 wt. % witch hazel extract.

15. Composition according to claim 13, for use in the treatment and/or prevention of a condition selected from a condition related to pro-oxidative and/or inflammatory activities in a human or animal.

16. Composition according to claim 13, taking a form selected from a tablet, cachet, capsule, granule, gelatin capsule, sugar-coated tablet, chewing gum, paste, beverage, syrup and powder.

17. Foodstuff, food supplement, dietary supplement, meal replacement, beverage, beverage supplement, or pharmaceutical product, comprising the composition according to claim 13.

18. Composition according to claim 1, for use in the treatment and/or prevention of a condition selected from a condition related to pro-oxidative and/or inflammatory activities in a human or animal.

19. Composition according to claim 1, taking a form selected from a tablet, cachet, capsule, granule, gelatin capsule, sugar-coated tablet, chewing gum, paste, beverage, syrup and powder.

20. Foodstuff, food supplement, dietary supplement, meal replacement, beverage, beverage supplement, or pharmaceutical product, comprising the composition according to claim 1.
